(19) United States
(12) Patent Application Publication
(10) Pub. No.: US 2025/0042930 A1
(43) Pub. Date: Feb. 6, 2025

(54) METHOD FOR PREPARING ARYL CARBON GLYCOSIDE COMPOUND INCLUDING GLIFLOZIN DRUG

(71) Applicant: SICHUAN UNIVERSITY, Chengdu (CN)

(72) Inventors: Dawen NIU, Chengdu (CN); Xia ZHANG, Chengdu (CN); Chen ZHANG, Chengdu (CN); Hao ZUO, Chengdu (CN); Hongxin ZENG, Chengdu (CN); Yanjing LI, Chengdu (CN); Jiaxi WANG, Chengdu (CN)

(21) Appl. No.: 18/912,836

(22) Filed: Oct. 11, 2024

Related U.S. Application Data
(63) Continuation-in-part of application No. PCT/CN2022/118050, filed on Sep. 9, 2022.

(30) Foreign Application Priority Data
May 6, 2022 (CN) 202210488208.7

(51) Int. Cl. C07H 7/04

(52) U.S. Cl. CPC C07H 7/04

(57) ABSTRACT

In a method for preparing an aryl carbon glycoside compound including a gliflozin drug, a sulfinate glycosyl donor represented by formula W is used as a raw material to prepare the aryl carbon glycoside compound including the gliflozin drug. The method does not need to use an additional initiator.

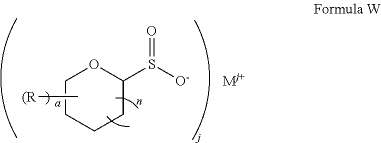

### TECHNOLOGY OF THE INVENTION

The present invention relates to the field of pharmaceutical chemistry, and specifically to a method for preparing aryl C-glycosides including gliflozins.

### BACKGROUND OF THE INVENTION

Carbohydrates are important components of living organisms (including animals, plants, and microorganisms). Polysaccharides, oligosaccharides, and their glycoconjugates with proteins, esters, etc. involve all temporal and spatial processes of cells, especially multicellular lives. They act as informational molecules and participate in various recognition processes of cells: transmitting biological information and participating in immune regulation of the body, and are closely related to various functions such as cell differentiation, fertilization, embryonic development, blood system, infection, aging, etc. In recent years, due to the significant physiological activity of carbohydrates, they have increasingly attracted the research interest of chemists. Glycosides are important forms of sugars that exist in nature. They are widely present in living organisms, have special biological activities, and play important physiological functions. Glycosides are a class of very important compounds formed by the condensation of the semi-acetal hydroxyl of a sugar with a ligand via the loss of one molecule of water or other small molecule compounds, in which the sugar part is called the glycosyl, and the non-sugar part is called the ligand. According to the type of atom in the molecular structure of glycoside compounds, by which the ligand is linked to the carbon of the sugar ring, glycoside compounds can be divided into O-glycosides, N-glycosides, S-glycosides, and C-glycosides. Most of them exhibit good biological functions, such as glycosidase inhibition, antibacterial, antiviral, and anti-tumor activities.

The international patent application with application number WO2021013155A1 discloses an allylsulfone glycosyl donor and a method for preparing S-glycosides, O-glycosides, and C-glycosides using the allylsulfone glycosyl donor as starting materials, for example, a method for preparing aryl C-glycosides from the allylsulfone glycosyl donor and tetrafluoroborate pyridine salt (synthesis route being as follows). This method involves adding a glycosyl donor 3-1 (1.0 equiv), a glycosyl acceptor pyridinium tetrafluoroborate (2.0 equiv), a photosensitizer Eosin Y (0.025 equiv.), and an initiator sodium trifluoromethylsulfite (0.2 equiv.) to a catalytic reaction flask under nitrogen atmosphere, to which is added DMSO, and then stirring at room temperature for 8 h under the irradiation of Blue LED, to obtain aryl C-glycoside compound C-X.

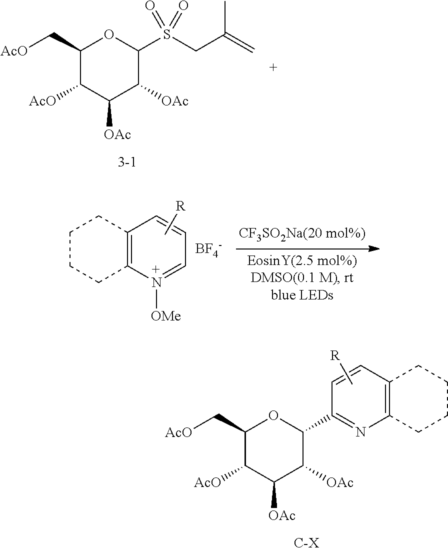

However, when the allylsulfone glycosyl donor is used to prepare aryl C-glycosides, an additional initiator sodium trifluoromethylsulfinate is required to induce the generation of sugar free radical intermediates, which has the following problems: 1. The additional initiator used increases the cost of the reaction; 2. It makes the reaction conditions more complex, and the compatibility worse; 3. It will generate additional by-products and make the separation of product more complicated, thereby difficult to obtain high-purity of aryl C-glycosides. In another aspect, this method can only be used to prepare pyridine C-glycosides, while cannot be used to prepare gliflozins.

As a class of hypoglycemic medicaments, gliflozins can reduce glucose reabsorption in the kidney and increase urine glucose excretion (UGE) by highly selective inhibition on SGLT-2 (sodium glucose cotransporter 2), thus reducing blood sugar in patients with type II diabetes. Currently, gliflozins have become the first choice for the treatment of type II diabetes. Nowadays, there are 9 gliflozins on the market worldwide, and 4 of them have been approved for market in China, namely dapagliflozin, canagliflozin, empagliflozin, and ertugliflozin. Among them, empagliflozin was commercially available in 2014, and its global sales reached over 3 billion US dollars in 2019.

For gliflozins, the currently reported methods include the following one for synthesizing empagliflozin, that was disclosed by Shi Ke-Jin et al. (Study on the synthesis of empagliflozin, Chemical Research and Application, November 2016, volume 28, issue 11): the intermediate (S)-4-bromo-1-chloro-2-(4-tetrahydrofuran-3-yloxy-benzyl)benzene was synthesized from 5-bromo-2-chlorobenzoic acid via chlorination, Friedel-Crafts acylation reaction, nucleophilic substitution, and reduction, and then the intermediate was condensed with 2,3,4,6-tetra-O-trimethylsilyl-D-glucono-1,5-lactone, followed by etherification and removal of the methoxy to obtain empagliflozin, with an overall yield of 29.8% and purity of 99.13%. As for this method, not only was the yield low, but also a reaction temperature of −78° C. was required in the synthesis procedures, and controlling the temperature is difficult and not conducive to industrial production. In order to improve the yield of gliflozins, researchers have modified the above process and reported an improved process for synthesizing empagliflozin (Chinese Journal of Pharmaceuticals, 2018, Volume 49, Issue 8): phenol (2) reacted with (R)-3-hydroxytetrahydrofuran to obtain (S)-3-phenoxytetrahydrofuran, which was allowed to react with 2-chloro-5-iodobenzyl bromide via Friedel-Crafts alkylation to produce (S)-3-[4-(2-chloro-5-iodobenzyl)phenoxy]tetrahydrofuran (6). In the presence of n-butyl lithium and CuI, 6 was condensed with 2,3,4,6-tetra-O-acetyl-1-α-bromo-D-glucopyranose to obtain (2S,3R,4R,5S,6R)-2-[3-[4-[(S)-tetrahydrofuran-3-yloxy]benzyl]-4-chlorophenyl]-6-acetoxymethyl-3,4,5-triacetoxyepoxyhexane (8); Finally, the target compound 1 (i.e. empagliflozin) was synthesized by deacetylation of 8 using lithium hydroxide, with a total yield of 43.0% (Based on 2) and a product purity of 99.21%. Although this method improves the yield of the product, it has the following problems: (1) the reaction route is lengthy, the economic cost increases, and the stability of the glycosyl donor used is poor. It is necessary to first protect the hydroxyl groups in the glycosyl donor with protective groups to prevent them from interfering with the key bonding step. After synthesizing compound 8, the protective group is removed to obtain the target product empagliflozin; (2) The reaction temperature at −40° C. is quite demanding and has high requirements on equipment; (3) The overall yield needs to be further improved.

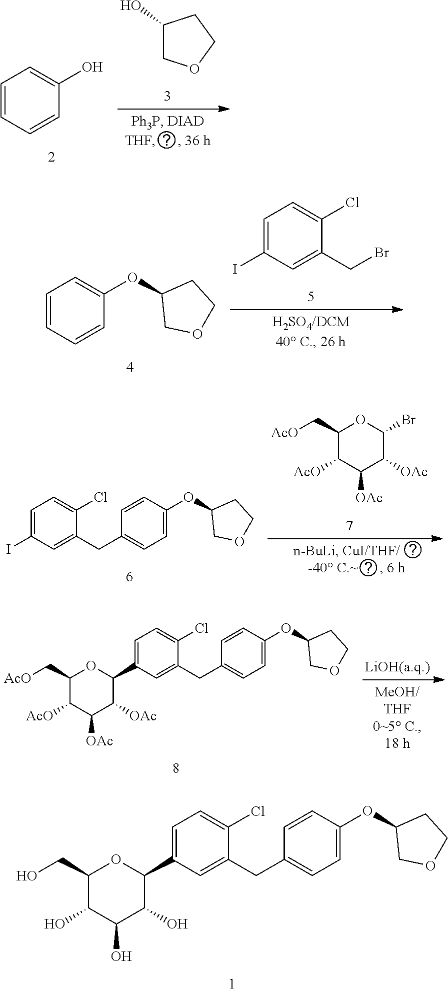

Therefore, developing a new method with a simple reaction route, lower production cost, mild reaction conditions, and no need for additional initiators is of great significance for the preparation of aryl C-glycosides, including gliflozins.

### CONTENT OF THE INVENTION

The object of the present invention is to provide a new method for preparing aryl C-glycosides including gliflozins.

The present invention provides a method for preparing aryl C-glycosides, which comprises the following steps: a glycosyl donor represented by formula W, a glycosyl acceptor represented by formula B-1, Ni(II) catalyst, a photosensitizer, a ligand, and a base are added to a solvent, and then allowed to react under light, to obtain an aryl C-glycoside represented by formula B-2;

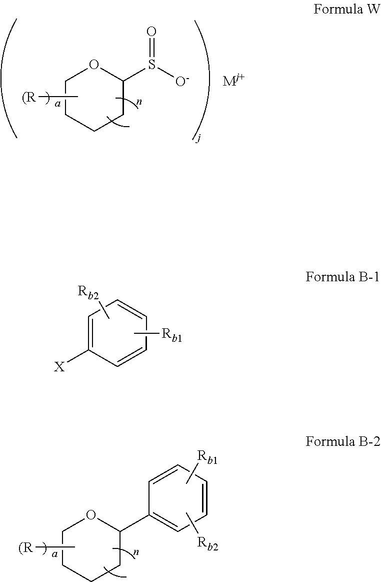

wherein, n is selected from 0 or 1;
a is selected from 3 or 4;
R is each independently selected from L₁Rₓ; alternatively, two adjacent R are linked to form a ring, and additional R is independently selected from L₁Rₓ, wherein the ring is unsubstituted or substituted with one or more L₁Rₓ;
L₁ is selected from absence or C₁₋₂ alkylene; Rx is selected from the group consisting of H, hydroxyl, C₁₋₆ alkyl, OAc, OBn, OR₈, NR₉R₁₀,

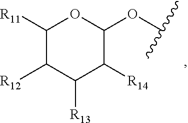

Ph, amino,

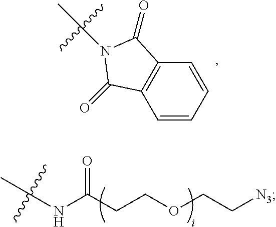

i is an integer selected from 0 to 6;
R₁₁, R₁₂, R₁₃, and R₁₄ are each independently selected from L₂R_{y}; L₂ is selected from absence or C₁₋₂ alkylene, and R_{y} is selected from the group consisting of H, hydroxyl, C₁₋₆ alkyl, OAc, OBn, OR₈, and NR₉R₁₀;
R₈ is selected from C₁₋₆ alkyl;
R₉ is selected from the group consisting of H, C₁₋₆ alkyl, Ac, and Bn;
R₁₀ is selected from the group consisting of H, C₁₋₆ alkyl, Ac, and Bn;
j is 1, 2, or 3;
M^{j+} is a j-valent cation;
X is halogen;
R_{b1} is selected from the group consisting of H, hydroxyl, C₁₋₆ alkyl, C₁₋₆ alkoxy, and halogen;
R_{b2} is selected from the group consisting of H and

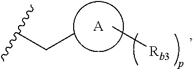

wherein ring A is selected from the group consisting of 5-6-membered aryl, 5-6 membered heteroaryl, fused cycloalkyl, or fused heterocyclic group; p is an integer from 0 to 2; R_{b3} is each independently selected from the group consisting of C₁₋₄ alkyl, C₁₋₄ alkoxy, halogenated or unhalogenated phenyl, and OR_{b4}, and R_{b4} is selected from 5-6-membered saturated heterocyclic group or 5-6 membered saturated cycloalkyl.

Further, the structure of the glycosyl donor is as represented by formula I.

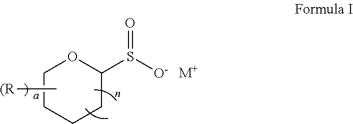

wherein, n is selected from 0 or 1;
a is selected from 3 or 4;
R is each independently selected from L₁Rₓ; alternatively, two adjacent R are linked to form a ring, and the remaining R is independently selected from L₁Rₓ, wherein the ring is unsubstituted or substituted with one or more L₁Rₓ;
L₁ is selected from absence or C₁₋₂ alkylene; Rₓ is selected from the group consisting of H, hydroxyl, C₁₋₆ alkyl, OAc, OBn, OR₈, NR₉R₁₀, R₁₃,

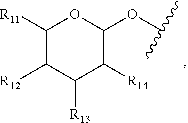

Ph, amino,

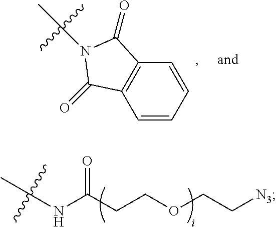

i is an integer selected from 0 to 6;
R₁₁, R₁₂, R₁₃, and R₁₄ are each independently selected from L₂R_{y}; L₂ is selected from absence or C₁₋₂ alkylene, and R_{y} is selected from the group consisting of H, hydroxyl, C₁₋₆ alkyl, OAc, OBn, OR₈, and NR₉R₁₀;
R₈ is selected from C₁₋₆ alkyl;
R₉ is selected from the group consisting of H, C₁₋₆ alkyl, Ac, and Bn;
R₁₀ is selected from the group consisting of H, C₁₋₆ alkyl, Ac, and Bn;
M^{j+} is a monovalent cation.

Further, the structure of the glycosyl donor is as represented by formula II:

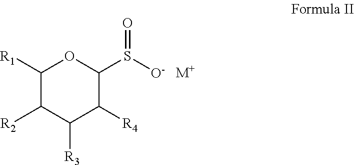

wherein, R₁, R₂, R₃, and R₄ are each independently selected from L₁Rₓ; alternatively, for R₁, R₂, R₃, and R₄, two adjacent groups are linked to form a ring, and two remaining groups are each independently selected from L₁Rₓ. The ring is a 5-6-membered ring which is unsubstituted or substituted with one or more L₁Rₓ;
L₁ is selected from absence or methylene, and Rₓ is selected from the group consisting of H, OH, C₁₋₅ alkyl, OAc, OBn, OR₈, NR₉R₁₀,

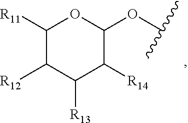

Ph, amino,

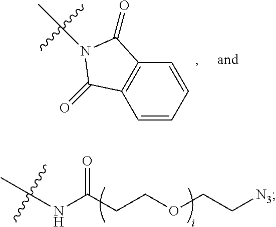

i is an integer selected from 0 to 4;
R₁₁, R₁₂, R₁₃, and R₁₄ are each independently selected from L₂R_{y}; L₂ is selected from absence or methylene, and R_{y} is selected from the group consisting of H, hydroxyl, C₁₋₅ alkyl, OAc, OBn, OR₈, and NR₉R₁₀;
R₈ is selected from C₁₋₅ alkyl;
R₉ is selected from the group consisting of H, C₁₋₅ alkyl, Ac, and Bn;
R₁₀ is selected from the group consisting of H, C₁₋₅ alkyl, Ac, and Bn;
M^{j+} is a monovalent cation.

Further, the structure of the glycosyl donor is as represented by formula III:

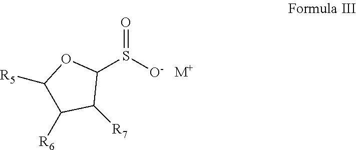

wherein, R₅, R₆, and R₇ are each independently selected from L₁Rₓ; alternatively, for R₅, R₆, and R₇, two adjacent groups are linked to form a ring, and the remaining group is L₁Rₓ. The ring is a 5-6-membered ring that is unsubstituted or substituted with one or more L₁Rₓ;
L₁ is selected from absence or methylene, and Rₓ is selected from the group consisting of H, OH, C₁₋₅ alkyl, OAc, OBn, OR₈, NR₉R₁₀,

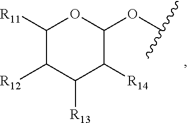

Ph, amino,

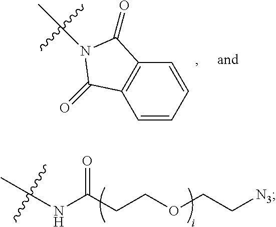

i is an integer selected from 0 to 4;
R₁₁, R₁₂, R₁₃, and R₁₄ are each independently selected from L₂R_{y}; L₂ is selected from absence or methylene, and R_{y} is selected from the group consisting of H, hydroxyl, C₁₋₅ alkyl, OAc, OBn, OR₈, and NR₉R₁₀;
R₈ is selected from C₁₋₅ alkyl;
R₉ is selected from the group consisting of H, C₁₋₅ alkyl, Ac, and Bn;
R₁₀ is selected from the group consisting of H, C₁₋₅ alkyl, Ac, and Bn;
M^{j+} is a monovalent cation.

Further, the 5-6-membered ring is a 5-6-membered saturated oxygen-containing heterocycle;
R₈ is selected from C₁₋₃ alkyl;
R₉ is selected from the group consisting of H, C₁₋₃ alkyl, Ac, and Bn;
R₁₀ is selected from the group consisting of H, C₁₋₃ alkyl, Ac, and Bn;
M⁺ is selected from the group consisting of Na⁺, K⁺, and Li⁺.

Further, the structure of the glycosyl donor is selected from the group consisting of:

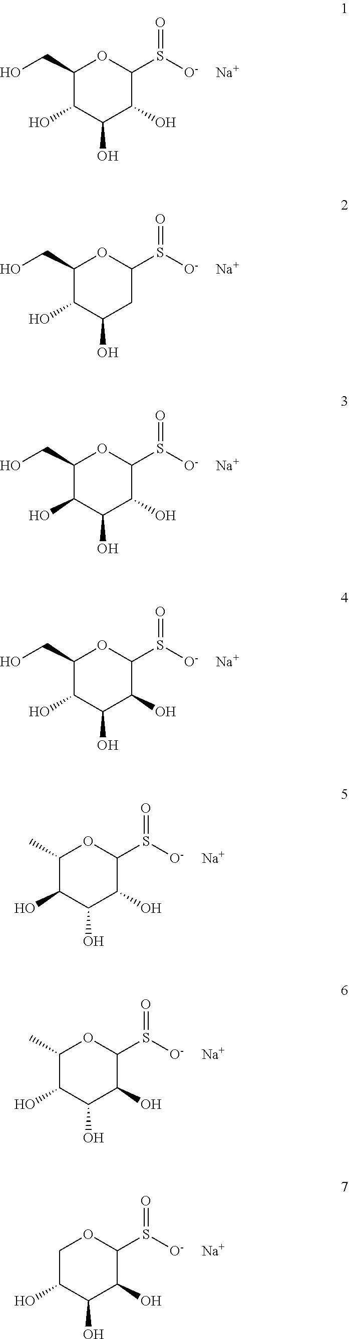

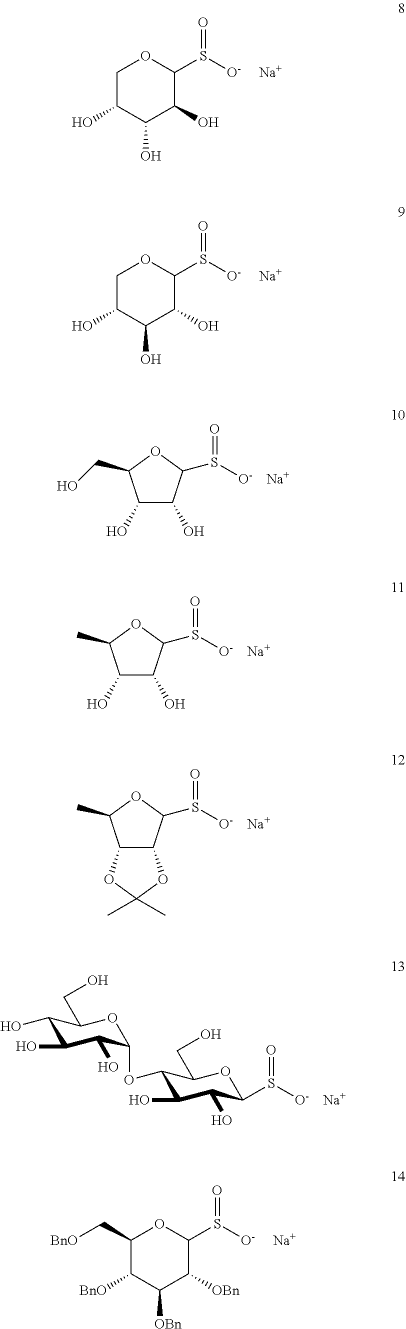

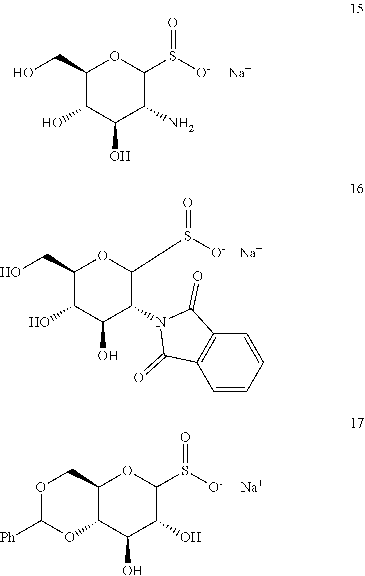

Further, the molar ratio of the glycosyl donor, glycosyl acceptor represented by formula B-1, photosensitizer, Ni(II) catalyst, ligand, and base is (0.07-0.3):(0.05-0.2):(5-20):(50-200):(10-240):(0.1-0.4); preferably 0.15:0.1:10:100:(50-120):0.2; and more preferably 0.15:0.1:10:100:120:0.2; and/or, said photosensitizer is selected from Ru(II) photosensitizers, eosin Y or salts thereof, wherein the Ru (II) photosensitizer is preferably Ru(bpy)₃Cl₂·6H₂O, and the salt of eosin Y is preferably Eosin Y/Na⁺;
and/or, said Ni(II) catalyst is NiBr₂·DME, NiBr₂, NiCl₂, Ni(OAc)₂ or Ni(acac)₂, and preferably NiBr₂·DME;
and/or, said ligand is N,N ligand, and preferably diOMebpy;

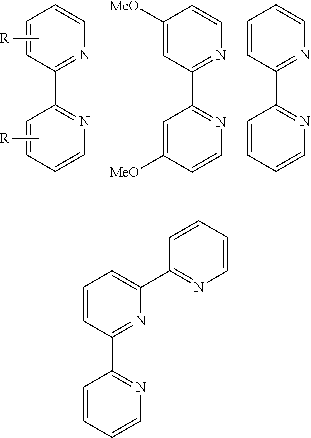

and/or, said base is an organic or inorganic base, and preferably tetramethylguanidine;
and/or, said solvent is an organic solvent; preferably DMSO, DMF, and DMA; and more preferably DMSO;
and/or, the reaction is carried out under an inert gas atmosphere; the reaction temperature is room temperature, and the reaction time is 4-14 h;
and/or, said X is iodine or bromine.

diOMebpy is chemically named 4,4′-dimethoxy-2,2′-bipyridine, and has a structure of

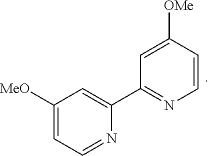 [SMILES: COc1ccnc(-c2cc(OC)ccn2)c1]

Further, the above light is performed by an LED lamp, with parameters of 10 W and 455 nm.

Further, the aryl C-glycosides represented by formula B-2 are

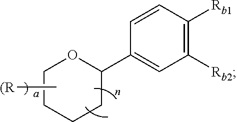

R_{b1} is selected from the group consisting of H, C₁₋₃ alkyl, C₁₋₃ alkoxy, and halogen; R_{b2} is selected from the group consisting of H,

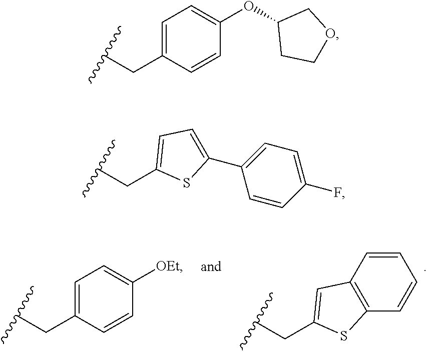 [SMILES: *Cc1ccc(O[C@H]2CCOC2)cc1 ; *Cc1ccc(-c2ccc(F)cc2)s1 ; *Cc1ccc(OCC)cc1 ; *Cc1cc2ccccc2s1]

Further, the aryl C-glycosides represented by formula B-2 are selected from the group consisting of:

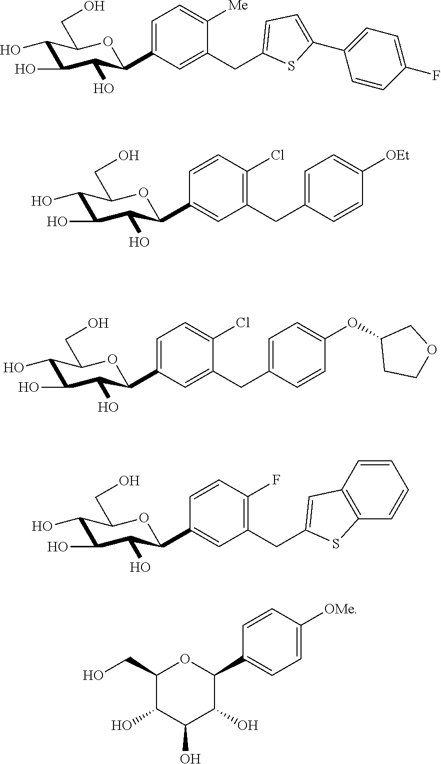

Further, the reaction device is that for a stirring reaction or flow chemistry.

Furthermore, provided that the reaction device is a stirring reaction device, the reaction time is 10-14 hours, and preferably 12 hours; provided that the reaction device is a flow chemistry device, the reaction time is 4-5 hours.

Glycosyl donor refers to the starting material that contains a glycosidic bond during the synthesis of glycosides, or the starting material that contains an anomeric carbon joining in the reaction; and another starting material that reacts with it is called a glycosyl receptor.

The sulfinate glycosyl donor provided in the present invention has a novel structure containing a special sulfinate moiety. The method for preparing the sulfinate glycosyl donor is simple, the reaction conditions are mild, and the yield is high, indicating the method is suitable for industrial production.

Aryl glycosides including gliflozins are first prepared using the sulfinate glycosyl donor of the present invention as the starting materials. This method does not require additional initiators; features low production costs, a simple reaction route, mild reaction conditions, high product yield and purity; and shows broad application prospects.

The method of the present invention for preparing aryl C-glycosides (including gliflozins) can be carried out not only in conventional devices for stirring reactions but also in flow chemistry devices. When carried out in a flow chemistry device, continuous-flow synthesis of aryl C-glycosides can be achieved, further shortening reaction time and improving production efficiency.

For the definition of terms used in the present invention: unless defined otherwise, the initial definition provided for the group or term herein applies to the group or term of the whole specification; for the terms that are not specifically defined herein, based on the disclosed content and context, they should have the same meaning as commonly understood by one of ordinary skill in the art to which the disclosure belongs.

The minimum and maximum values of carbon atom content in the hydrocarbon group are indicated by a prefix, for example, the prefix C_{a-b} alkyl indicates any alkyl having “a” to “b” carbon atoms. For example, C₁₋₈ alkyl means a straight or branched alkyl containing 1-8 carbon atoms.

Similarly, C₁₋₆ alkoxy means a straight or branched alkoxy containing 1-6 carbon atoms.

For the groups of the present invention, Ac represents acetyl; Bn represents benzyl; Ph represents phenyl; Me represents methyl.

“m-CPBA” is m-chloro-peroxybenzoic acid.

“Salts” are acid and/or basic salts formed by compounds with inorganic and/or organic acids and/or bases, also including amphoteric salts (inner salts) as well as quaternary ammonium salts (e.g. alkylammonium salts). These salts can be directly obtained in the final isolation and purification of the compound, and can also be obtained by mixing the compound with a certain amount of acid or base (e.g., equivalent). These salts may form a precipitate in the solution and be collected by filtration, or recovered after evaporation of the solvent, or prepared by freeze-drying after reacting in an aqueous medium.

In the present invention, the salt may be hydrochloride, sulfate, citrate, benzenesulfonate, hydrobromide, hydrofluoride, phosphate, acetate, propionate, succinate, oxalate, malate, succinate, fumarate, maleate, tartrate or trifluoroacetate of the compound.

Obviously, based on the above content of the present invention, according to the common technical knowledge and the conventional means in the field, other various modifications, alternations, or changes can further be made, without department from the above basic technical spirits.

By following description of specific examples, the above content of the present invention is further illustrated. But it should not be construed that the scope of the above subject matter of the present invention is limited to the following examples. The techniques realized based on the above content of the present invention are all within the scope of the present invention.

### EXAMPLES

The starting materials and equipment used in the examples of the present invention are all known products, which are obtained by purchasing those commercially available.

The sodium sulfinate glycosyl donor of the present invention was prepared using the following synthetic routes:

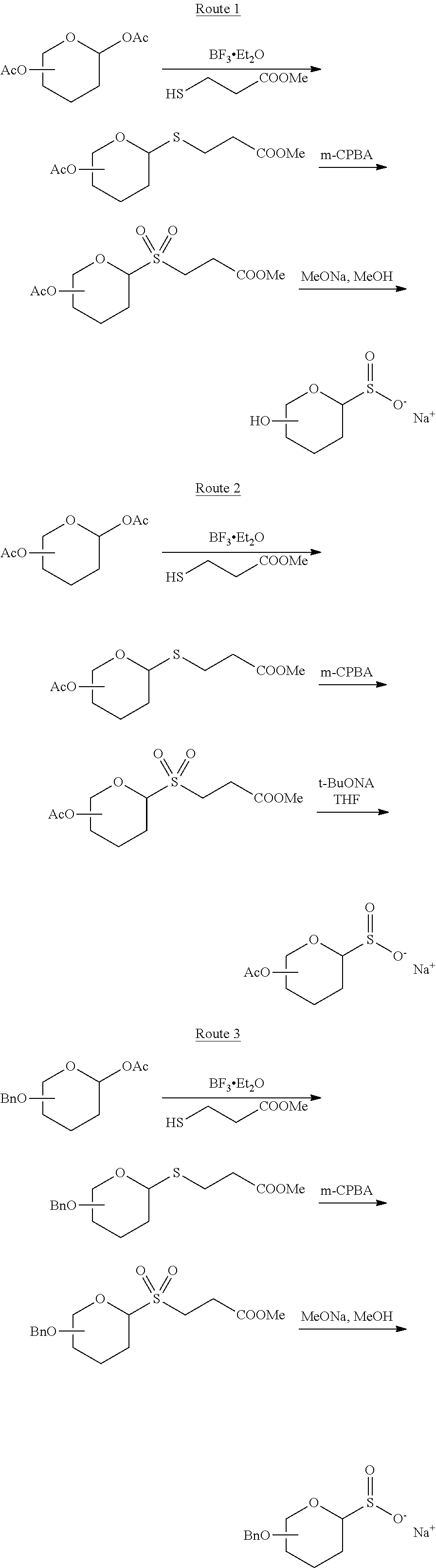

### Example 1: Preparation of Sodium Sulfinate Glycosyl Donors 1-20

### I. Preparation of Sodium Sulfinate Glycosyl Donor 1

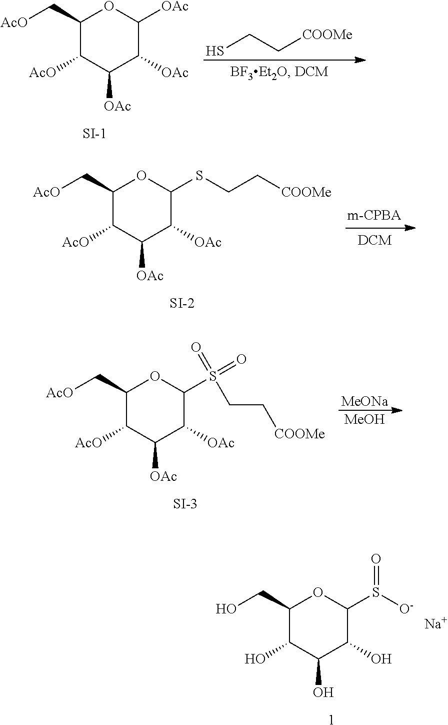

Specific procedures were as follows:

Step 1: To a 100 mL round bottom flask containing SI-1 (3.9 g, 10 mmol, 1.0 equiv) and 25 mL of CH₂Cl₂, were added methyl 3-mercaptopropionate (1.3 mL, 12 mmol, 1.2 equiv) and BF₃·Et₂O (2.5 mL, 20 mmol, 2.0 equiv) sequentially. The reaction solution was stirred at room temperature for 1 h, until SI-1 was completely disappeared by TLC detection, and then washed with saturated NaHCO₃ aqueous solution to be neutral. The organic layers were separated, washed with saline, dried over anhydrous Na₂SO₄, and then concentrated to obtain SI-2, which could be directly used for the next step without purification.

Step 2: SI-2 was dissolved in 20 mL of CH₂Cl₂ and then cooled at 0° C. m-CPBA (m-chloroperoxybenzoic acid, 6 g, 30 mmol, 3 equiv) was slowly added to the reaction solution under stirring. The mixed solution was stirred at room temperature for 1 h and filtered. The filtrate was washed with saturated NaHCO₃ solution until neutral, dried over anhydrous Na₂SO₄, and concentrated. Methyl tert-butyl ether was added to precipitate the solid, which was collected by filtration to obtain SI-3 as white solid.

Step 3: SI-3 was dissolved in 20 mL of MeOH at 0° C., to which was added MeONa (540 mg, 10 mmol, 1.0 equiv), and then the reaction was stirred at 0° C. for 2 h. TLC detection indicated that SI-3 was completely consumed before concentration. The residue was washed with absolute ethanol, and then the resultant solution was filtered to obtain white solid, namely sodium sulfinate glycosyl donor 1. The total yield for three steps was 85%.

### II. Preparation of Sodium Sulfinate Glycosyl Donors 2-20

Sodium sulfinate glycosyl donors 2-20 were separately prepared by referring to the above method for preparing sodium sulfinate glycosyl donor 1, with the only difference being that the starting material SI-1 was substituted with the corresponding starting materials, respectively.

The structure and characterization of sodium sulfinate glycosyl donors 1-20 are shown in Table 1. The total yield for three steps and the purity of sodium sulfinate glycosyl donors 1-20 are shown in Table 2.

TABLE 1
The structure and characterization of sodium sulfinate glycosyl donors 1-20.

| | | Starting materials | Structural |
|---|---|---|---|
| No. | Structure | corresponding to SI-1 | characterization |
| 1 | 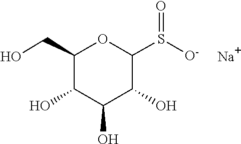 [SMILES: O=S([O-])C1O[C@H](CO)[C@@H](O)[C@H](O)[C@H]1O ; [Na+]] | 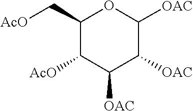 | ¹H NMR (400 MHz, CD₃OD) δ 3.85 (d, J = 12.9 Hz, 1H), 3.73 (t, J = 9.3 Hz, 1H), 3.66 (dd, J = 12.0, 5.2 Hz, 1H), 3.43 (t, J = 8.4 Hz, 1H), 3.35 (d, J = 9.7 Hz, 1H), 3.30 (d, J = 5.3 Hz, 2H); ¹³C NMR (101 MHz, D₂O) δ 92.68, 79.99, 77.10, 69.76, 69.16, 60.94. |
| 2 | 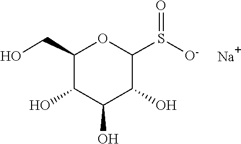 [SMILES: O=S([O-])C1O[C@H](CO)[C@@H](O)[C@H](O)[C@H]1O ; [Na+]] | 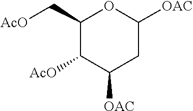 | ¹³C NMR (101 MHz, D₂O) δ 96.29, 76.96, 70.67, 68.55, 60.78, 31.38. |
| 3 | 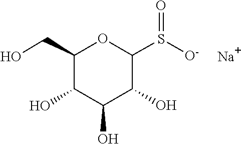 [SMILES: O=S([O-])C1O[C@H](CO)[C@@H](O)[C@H](O)[C@H]1O ; [Na+]] | 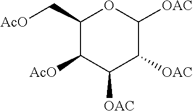 | ¹H NMR (400 MHz, CD₃OD) δ 3.99 (t, J = 9.5 Hz, 1H), 3.83 (dd, J = 11.7, 7.7 Hz, 1H), 3.78 (d, J = 3.3 Hz, 1H), 3.66- 3.60 (m, 1H), 3.58 (q, J = 3.9 Hz, 1H), 3.53 (dd, J = 9.4, 3.4 Hz, 1H), 3.34 (d, J = 3.5 Hz, 1H). ¹³C NMR (101 MHz, D₂O) δ 93.72, 79.49, 73.96, 69.08, 67.06, 61.60. |
| 4 | 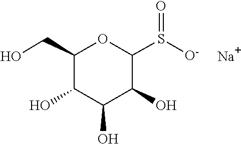 [SMILES: O=S([O-])C1O[C@H](CO)[C@@H](O)[C@H](O)[C@@H]1O ; [Na+]] | 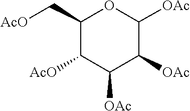 [SMILES: CC(=O)OC[C@H]1OC(OC(C)=O)[C@@H](OC(C)=O)[C@@H](OC(C)=O)[C@@H]1OC(C)=O] | ¹H NMR (400 MHz, D₂O) δ 4.22 (d, J = 3.0 Hz, 1H), 3.78 (m, 2H), 3.71-3.62 (m, 3H), 3.50 (d, J = 1.3 Hz, 1H); ¹³C NMR (101 MHz, D₂O) δ 100.65, 77.55, 71.25, 67.94, 66.20, 61.06 |
| 5 | 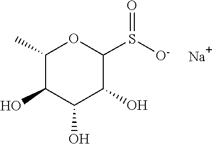 | 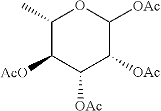 [SMILES: CC(=O)OC1O[C@@H](C)[C@H](OC(C)=O)[C@@H](OC(C)=O)[C@H]1OC(C)=O] | ¹H NMR (400 MHz, D₂O) δ 4.33-4.29 (m, 1H), 4.20-4.10 (m, 1H), 3.85-3.76 (m, 2H), 3.52- 3.45 (m, 1H), 1.31 (d, J = 6.1, 3H). ¹³C NMR (101 MHz, D₂O) δ 90.32, 71.66, 69.90, 68.21, 57.41, 16.76. |
| 6 | 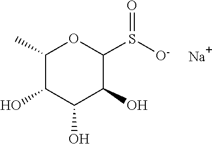 | 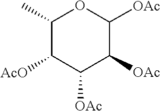 [SMILES: CC(=O)OC1O[C@@H](C)[C@@H](OC(C)=O)[C@@H](OC(C)=O)[C@@H]1OC(C)=O] | ¹H NMR (400 MHz, D₂O) δ 3.86-3.80 (m, 1H), 3.72 (d, J = 6.3 Hz, 1H), 3.62 (dt, J = 10.6, 2.9 Hz, 1H), 3.56 (td, J = 7.1, 1.9 Hz, 1H), 3.39 (dd, J = 9.7, 1.9 Hz, 1H), 1.22- 1.17 (m, 3H); ¹³C NMR (101 MHz, D₂O) δ 93.75, 75.19, 74.10, 71.61, 66.57, 16.78. |
| 7 | 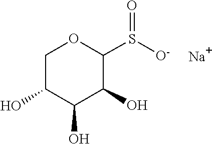 [SMILES: O=S([O-])C1OC[C@@H](O)[C@H](O)[C@@H]1O ; [Na+]] | 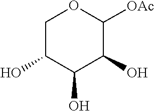 [SMILES: CC(=O)OC1OC[C@@H](O)[C@H](O)[C@@H]1O] | ¹³C NMR (101 MHz, D₂O) δ 99.50, 73.85, 70.79, 69.85, 68.38. |
| 8 | 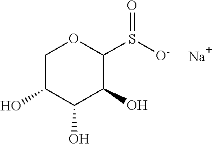 [SMILES: O=S([O-])C1OC[C@@H](O)[C@@H](O)[C@@H]1O ; [Na+]] | 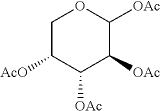 [SMILES: CC(=O)OC1OC[C@@H](OC(C)=O)[C@@H](OC(C)=O)[C@@H]1OC(C)=O] | Mixture of a and b□¹H NMR (400 MHz, D₂O) δ 4.37 (td, J = 3.9, 1.3 Hz, 0.5 H), 4.03-3.83 (m, 3H), 3.75-3.54 (m, 2.5H); ¹³C NMR (101 MHz, D₂O) δ 96.71, 72.41, 71.81, 70.31, 68.44 |
| 9 | 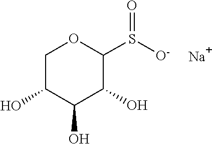 [SMILES: O=S([O-])C1OC[C@@H](O)[C@H](O)[C@H]1O ; [Na+]] | 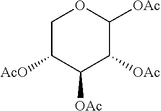 [SMILES: CC(=O)OC1OC[C@@H](OC(C)=O)[C@H](OC(C)=O)[C@H]1OC(C)=O] | ¹H NMR (400 MHz, D₂O) δ 3.98 (dd, J = 11.1, 5.3 Hz, 1H), 3.60 (t, J = 9.2 Hz, 1H), 3.52 (td, J = 9.7, 5.2 Hz, 1H), 3.42 (t, J = 9.2 Hz, 2H), 3.22 (t, J = 10.8 Hz, 1H); ¹³C NMR (101 MHz, D₂O) δ 93.61, 77.12, 69.54, 69.15, 68.86. |
| 10 | 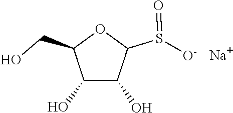 [SMILES: O=S([O-])C1O[C@H](CO)[C@@H](O)[C@H]1O ; [Na+]] | 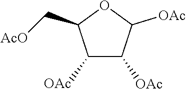 [SMILES: CC(=O)OC[C@H]1OC(OC(C)=O)[C@H](OC(C)=O)[C@@H]1OC(C)=O] | Mixture of α:β = 5:4, ¹H NMR (400 MHz, D₂O): δ 4.43 (t, J = 4.2 Hz, 0.43H), 4.29 (dd, J = 4.7, 2.1 Hz, 1H), 4.05 (dd, J = 8.4, 4.5 Hz, 0.55H), 4.00- 3.91 (m, 2.46H), 3.88- 3.68 (m, 2.94H), and 3.58 (td, J = 12.4, 4.7 Hz, 1.74H); ¹³C NMR (101 MHz, D₂O): δ 102.18, 100.63, 83.01, 82.53, 71.53, 71.30, 71.16, 70.70, 61.70, 60.76. |
| 11 | 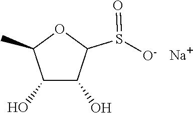 | 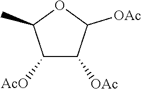 [SMILES: CC(=O)OC1O[C@H](C)[C@@H](OC(C)=O)[C@H]1OC(C)=O] | ¹H NMR (400 MHz, Methanol-d₄) δ 4.28 (d, J = 5.0 Hz, 1H), 3.96 (t, J = 6.3 Hz, 1H), 3.81 (t, J = 1.8 Hz, 1H), 3.73-3.65 (m, 1H), 1.21 (d, J = 6.3 Hz, 3H); ¹³C NMR (101 MHz, D₂O) δ 101.85, 78.88, 76.35, 70.51, 17.67. |
| 12 | 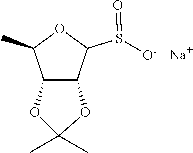 | 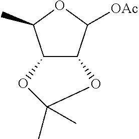 | ¹H NMR (400 MHz, CD₃OD) δ 5.01 (t, J = 4.5 Hz, 1H), 4.45 (d, J = 6.4 Hz, 1H), 4.20 (q, J = 7.1 Hz, 1H), 3.62 (dd, J = 4.4, 1.7 Hz, 1H), 1.46 (s, 3H), 1.30 (s, 3H), 1.15 (d, J = 5.1 Hz, 3H). |
| 13 | 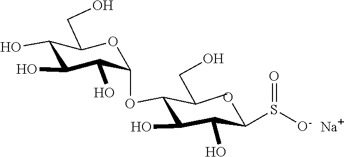 | 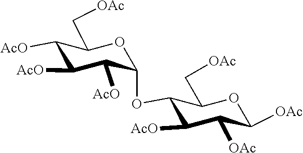 | ¹H NMR (400 MHz, D₂O) δ 5.32 (d, J = 3.8 Hz, 1H), 3.86 (d, J = 11.7 Hz, 1H), 3.81-3.44 (m, 11H), 3.33 (t, J = 9.4 Hz, 1H); ¹³C NMR (101 MHz, D₂O) δ 99.67, 92.47, 78.56, 77.51, 76.44, 72.83, 72.66, 71.73, 69.62, 69.30, 60.94, 60.43. |
| 14 | 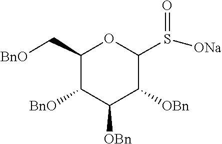 | 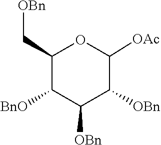 | ¹H NMR (400 MHz, CDCl₃) δ 7.36-7.27 (m, 14H), 7.26-7.22 (m, 4H), 7.15-7.06 (m, 2H), 4.93 (d, J = 11.1 Hz, 1H), 4.89 (d, J = 11.1 Hz, 1H), 4.81 (d, J = 10.7 Hz, 1H), 4.76 (d, J = 11.1 Hz, 1H), 4.68 (d, J = 10.6 Hz, 1H), 4.44 (d, J = 11.3 Hz, 1H), 4.41 (d, J = 11.1 Hz, 1H), 4.36 (d, J = 11.2 Hz, 1H), 4.11 (d, J = 9.7 Hz, 1H), 3.85 (t, J = 8.8 Hz, 1H), 3.77 (t, J = 9.4 Hz, 1H), 3.67 (ddt, J = 7.9, 4.8, 2.9 Hz, 2H), 3.60-3.54 (m, 1H), 3.51 (dd, J = 10.7, 5.4 Hz, 1H); ¹³C NMR (101 MHz, CDCl₃): δ 161.1, 160.7, 160.2, 159.8, 137.0, 136.5, 136.3, 135.3, 129.0, 128.9, 128.7, 128.7, 128.6, 128.6, 128.5, 128.4, 128.3, 128.0, 118.7, 115. 9, 113.1, 110.2, 92.0, 85.7, 78.4, 77.3, 77.0, 76.8, 76.7, 76.1, 75.6, 75.4, 73.8, 67.9 |
| 15 | 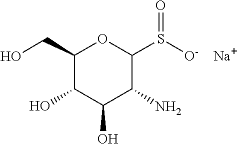 [SMILES: N[C@H]1C(S(=O)[O-])O[C@H](CO)[C@@H](O)[C@@H]1O ; [Na+]] | 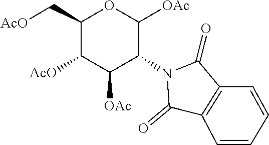 [SMILES: CC(=O)OC[C@H]1OC(OC(C)=O)[C@H](N2C(=O)c3ccccc3C2=O)[C@@H](OC(C)=O)[C@@H]1OC(C)=O] | ¹H NMR (400 MHz, D₂O) δ 3.92 (d, J = 13.0 Hz, 1H), 3.76 (dd, J = 12.5, 4.9 Hz, 1H), 3.71 (d, J = 10.3 Hz, 1H), 3.68- 3.60 (m, 1H), 3.48 (ddd, J = 22.8, 17.9, 9.0 Hz, 2H), 3.39-3.31 (m, 1H). |
| 16 | 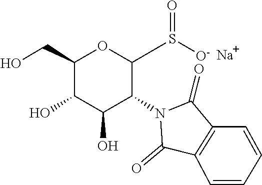 [SMILES: O=C1c2ccccc2C(=O)N1[C@H]1C(S(=O)[O-])O[C@H](CO)[C@@H](O)[C@@H]1O ; [Na+]] | 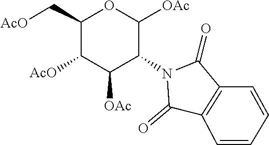 [SMILES: CC(=O)OC[C@H]1OC(OC(C)=O)[C@H](N2C(=O)c3ccccc3C2=O)[C@@H](OC(C)=O)[C@@H]1OC(C)=O] | ¹H NMR (400 MHz, MeOD) δ 7.84 (s, 2H), 7.77 (dd, J = 5.5, 3.1 Hz, 2H), 4.29 (dd, J = 6.7, 2.8 Hz, 2H), 4.13 (dd, J = 7.2, 3.3 Hz, 1H), 3.93 (dd, J = 12.1, 2.1 Hz, 1H), 3.73- 3.67 (m, 1H), 3.46 (ddd, J = 9.3, 6.7, 2.2 Hz, 1H), 3.34 (s, 1H). |
| 17 | 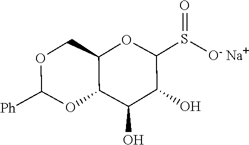 | 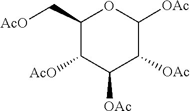 [SMILES: CC(=O)OC[C@H]1OC(OC(C)=O)[C@H](OC(C)=O)[C@@H](OC(C)=O)[C@@H]1OC(C)=O] | ¹H NMR (400 MHz, MeOD) δ 7.48 (dd, J = 6.8, 3.1 Hz, 2H), 7.41 (d, J = 2.5 Hz, 2H), 7.40- 7.39 (m, 1H), 5.68 (s, 1H), 4.31 (td, 1H), 3.82 (t, J = 10.2 Hz, 1H), 3.78- 3.72 (m, 2H), 3.60 (dqd, J = 9.3, 7.8, 7.3, 3.6 Hz, 3H). |
| 18 | 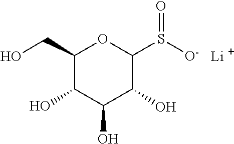 [SMILES: O=S([O-])C1O[C@H](CO)[C@@H](O)[C@H](O)[C@H]1O ; [Li+]] | 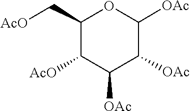 [SMILES: CC(=O)OC[C@H]1OC(OC(C)=O)[C@H](OC(C)=O)[C@@H](OC(C)=O)[C@@H]1OC(C)=O] | ¹H NMR (400 MHz, CD₃OD) δ 3.85 (d, J = 12.9 Hz, 1H), 3.73 (t, J = 9.3 Hz, 1H), 3.66 (dd, J = 12.0, 5.2 Hz, 1H), 3.43 (t, J = 8.4 Hz, 1H), 3.35 (d, J = 9.7 Hz, 1H), 3.30 (d, J = 5.3 Hz, 2H) |
| 19 | 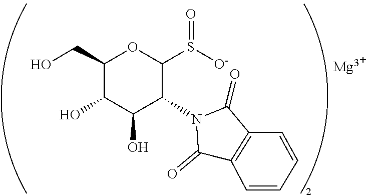 | 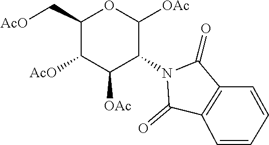 [SMILES: CC(=O)OC[C@H]1OC(OC(C)=O)[C@H](N2C(=O)c3ccccc3C2=O)[C@@H](OC(C)=O)[C@@H]1OC(C)=O] | ¹H NMR (400 MHz, CD₃OD) δ 7.83-7.76 (m, 1H), 7.55-7.50 (m, 1H), 7.47 (d, J = 4.5 Hz, 2H), 4.16 (t, J = 10.3 Hz, 1H), 3.90 (d, J = 12.1 Hz, 1H), 3.79-3.75 (m, 1H), 3.74- 3.67 (m, 11H), 3.36- 3.32 (m, 1H). |
| 20 | 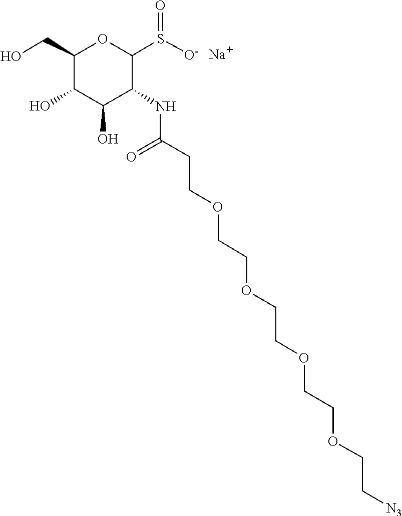 [SMILES: [N-]=[N+]=NCCOCCOCCOCCOCCC(=O)N[C@H]1C(S(=O)[O-])O[C@H](CO)[C@@H](O)[C@@H]1O ; [Na+]] | 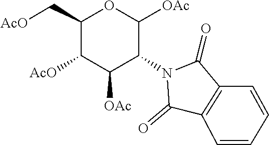 [SMILES: CC(=O)OC[C@H]1OC(OC(C)=O)[C@H](N2C(=O)c3ccccc3C2=O)[C@@H](OC(C)=O)[C@@H]1OC(C)=O] | ¹H NMR (400 MHz, D₂O) δ 3.94 (t, J = 10.3 Hz, 2H), 3.86-3.76 (m, 3H), 3.76-3.67 (m, 14H), 3.61 (d, J = 10.6 Hz, 2H), 3.54-3.50 (m, 2H), 3.49- 3.42 (m, 2H), 2.58 (t, J = 6.2 Hz, 2H). |

TABLE 2
The total yield for three steps and the purity of sodium sulfinate glycosyl donors 1-20.

| | | Total yield for | |
|---|---|---|---|
| No. | Structure | three steps | Purity |
| 1 | 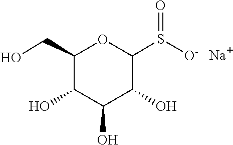 [SMILES: O=S([O-])C1O[C@H](CO)[C@@H](O)[C@H](O)[C@H]1O ; [Na+]] | 85% | 99% |
| 2 | 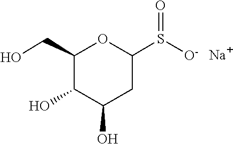 [SMILES: O=S([O-])C1C[C@@H](O)[C@H](O)[C@@H](CO)O1 ; [Na+]] | 77% | 95% |
| 3 | 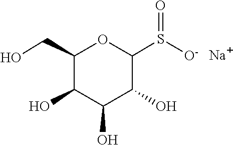 [SMILES: O=S([O-])C1O[C@H](CO)[C@H](O)[C@H](O)[C@H]1O ; [Na+]] | 87% | 98% |
| 4 | 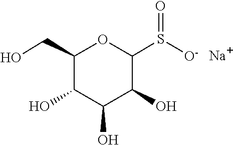 [SMILES: O=S([O-])C1O[C@H](CO)[C@@H](O)[C@H](O)[C@@H]1O ; [Na+]] | 75% | 98% |
| 5 | 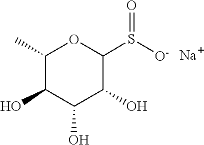 | 67% | 90% |
| 6 | 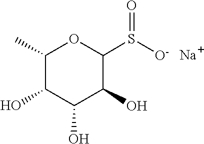 | 70% | 90% |
| 7 | 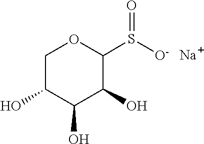 [SMILES: O=S([O-])C1OC[C@@H](O)[C@H](O)[C@@H]1O ; [Na+]] | 70% | 90% |
| 8 | 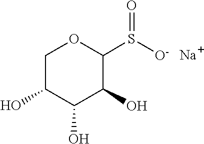 [SMILES: O=S([O-])C1OC[C@@H](O)[C@@H](O)[C@@H]1O ; [Na+]] | 70% | 90% |
| 9 | 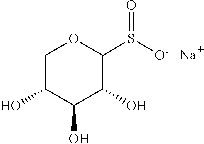 [SMILES: O=S([O-])C1OC[C@@H](O)[C@H](O)[C@H]1O ; [Na+]] | 70% | 90% |
| 10 | 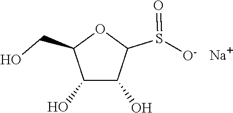 [SMILES: O=S([O-])C1O[C@H](CO)[C@@H](O)[C@H]1O ; [Na+]] | 65% | 85% |
| 11 | 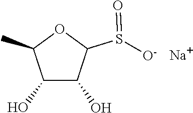 | 85% | 95% |
| 12 | 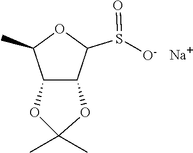 | 60% | 90% |
| 13 | 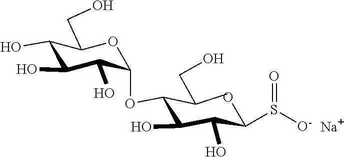 | 75% | 95% |
| 14 | 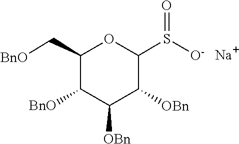 [SMILES: O=S([O-])C1O[C@H](COCc2ccccc2)[C@@H](OCc2ccccc2)[C@H](OCc2ccccc2)[C@H]1OCc1ccccc1 ; [Na+]] | 85% | 98% |
| 15 | 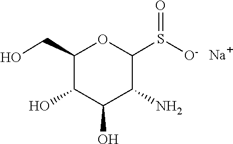 [SMILES: N[C@H]1C(S(=O)[O-])O[C@H](CO)[C@@H](O)[C@@H]1O ; [Na+]] | 82% | 98% |
| 16 | 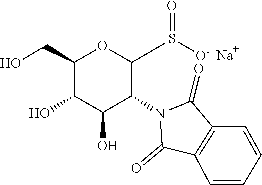 [SMILES: O=C1c2ccccc2C(=O)N1[C@H]1C(S(=O)[O-])O[C@H](CO)[C@@H](O)[C@@H]1O ; [Na+]] | 85% | 98% |
| 17 | 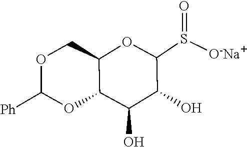 | 70% | 97% |
| 18 | 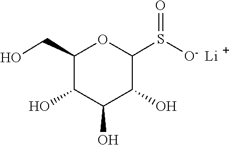 [SMILES: O=S([O-])C1O[C@H](CO)[C@@H](O)[C@H](O)[C@H]1O ; [Li+]] | 85% | 98% |
| 19 | 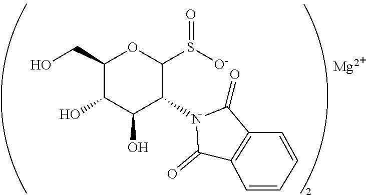 | 70% | 85% |
| 20 | 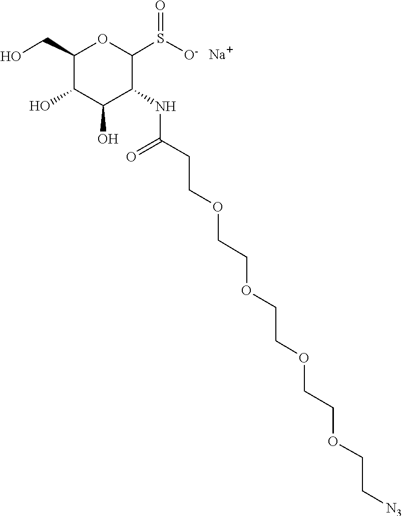 [SMILES: [N-]=[N+]=NCCOCCOCCOCCOCCC(=O)N[C@H]1C(S(=O)[O-])O[C@H](CO)[C@@H](O)[C@@H]1O ; [Na+]] | 57% | 98% |

### Example 2: Preparation of Aryl C-Glycosides Using Sodium Sulfinate Glycosyl Donors as Starting Materials

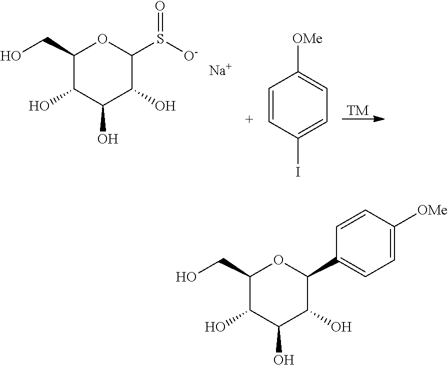

Aryl C-glycosides were synthesized according to the above route, and the specific procedures were as follows: Glycosyl donor 1 (0.15 mmol), 4-methoxyiodobenzene (0.1 mmol), Ru(bpy)₃Cl₂·6H₂O (0.8 mg, 1 mol %), NiBr₂·DME (3.1 mg, 10 mol %), diOMebpy (2.6 mg, 12 mol %), TMG (tetramethylguanidine, 12.5 μL, 0.2 mmol) and DMSO (0.5 mL) were weighed, transferred into a vial with a spiral cap and a magnetic stirring bar, and then mixed. The vial was filled with N₂ and sealed with a Teflon cap. The mixture was stirred at room temperature under a 10 W, 455 nm LED light for 12 h to complete the reaction.

The reaction solution was freeze-dried, and then the residue was subjected to column chromatography (C₁₈ chromatographic column, H₂O/MeCN=13:1 (v/v)), to obtain aryl glycosides as pure β-configuration, with a yield of 67% and a purity of >98%. The structural characterization was as follows:

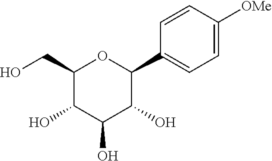 [SMILES: COc1ccc([C@@H]2O[C@H](CO)[C@@H](O)[C@H](O)[C@H]2O)cc1]

¹H NMR (400 MHz, DMSO-d₆) δ 7.25 (d, J=8.1 Hz, 2H), 6.86 (d, J=8.2 Hz, 2H), 4.98-4.83 (m, 2H), 4.70 (d, J=5.7 Hz, 1H), 4.42 (t, J=5.8 Hz, 1H), 3.95 (d, J=9.3 Hz, 1H), 3.82-3.59 (m, 4H), 3.44 (dt, J=11.8, 5.9 Hz, 1H), 3.30-3.08 (m, 4H); ¹³C NMR (101 MHz, DMSO-d₆) δ 158.59, 132.50, 128.97, 113.07, 81.16, 81.04, 78.49, 74.67, 70.45, 61.48, 55.05; HRMS (DART-TOF) calculated for C₁₃H₁₈NaO₆⁺ [M+Na]⁺ m/z 293.0996, found 293.0995.

(2) By referring to the above method, corresponding aryl C-glycosides were respectively synthesized by substituting glycosyl donor 1 with glycosyl donors 2-20.

The following examples 3-6 are specific examples for preparation of gliflozins using a traditional stirring reaction device.

### Example 3: Preparation of Gliflozins Using Sodium Sulfinate Glycosyl Donors as Starting Materials: Canagliflozin

### Method 1:

By referring to the method described in Example 2, except that 4-methoxyiodobenzene was substituted with 2-(4-fluorophenyl)-5-[(5-iodo-2-methylphenyl)methyl]thiophene, the hypoglycemic drug canagliflozin was prepared as pure β-configuration, with a yield of 54% and a purity of >98%. The structural characterization was as follows:

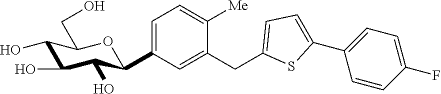

¹H NMR (400 MHz, CD₃OD) δ 7.54-7.48 (m, 2H), 7.31 (d, J=1.7 Hz, 1H), 7.24 (dd, J=7.8, 1.8 Hz, 1H), 7.15 (d, J=7.8 Hz, 1H), 7.08 (d, J=3.6 Hz, 1H), 7.07-7.01 (m, 2H), 6.68 (d, J=3.6 Hz, 1H), 4.16-4.12 (m, 2H), 4.11 (d, J=9.3 Hz, 1H), 3.88 (dd, J=11.9, 1.8 Hz, 1H), 3.70 (dd, J=11.9, 5.0 Hz, 1H), 3.51-3.35 (m, 4H), 2.29 (s, 3H).

### Method 2:

Glycosyl donor 1 (37.5 mg, 0.15 mmol), 2-(4-fluorophenyl)-5-[(5-iodo-2-methylphenyl) methyl]thiophene (0.1 mmol), Ru(bpy)₃Cl₂·6H₂O (0.8 mg, 1 mol %), NiBr₂·DME (3.1 mg, 10 mol %), diOMebpy (2.6 mg, 12 mol %), TMG (25 μL, 0.4 mmol), and DMSO (0.5 mL) were weighed, transferred into a vial with a spiral cap and a magnetic stirring bar, and then mixed. The vial was filled with N₂ and sealed with a Teflon cap. The mixture was stirred at room temperature under a 10 W, 455 nm LED light for 12 h to complete the reaction.

The reaction solution was diluted with 10 mL of ethyl acetate, and then washed with saturated NaCl solution. The organic phase was collected, and the aqueous phase was extracted three times with 6 mL of ethyl acetate. The organic phases were combined and concentrated under reduced pressure. The residue was separated by silica gel chromatography (using a solution of petroleum ether/ethyl acetate=10:5 (v/v) as the eluent), to obtain the product canagliflozin as pure β-configuration, with a yield of 55% and a purity of >95%.

### Example 4: Preparation of Gliflozins Using Sodium Sulfinate Glycosyl Donors as Starting Materials: Dapagliflozin

### Method 1:

By referring to the method described in Example 2, except that 4-methoxyiodobenzene was substituted with 1-chloro-2-(4-ethoxybenzyl)-4-iodobenzene, the hypoglycemic drug dapagliflozin was prepared as pure β-configuration, with a yield of 65% and a purity of >98%. The structural characterization was as follows:

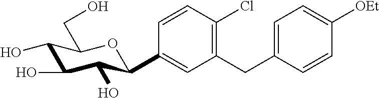

¹H NMR (400 MHz, CD₃CN) δ 7.37 (d, J=8.2 Hz, 1H), 7.30 (d, J=2.1 Hz, 1H), 7.24 (dd, J=8.2, 2.2 Hz, 1H), 7.12 (d, J=8.6 Hz, 2H), 6.86-6.80 (m, 2H), 4.08 (d, J=9.4 Hz, 1H), 4.05-4.02 (m, 2H), 3.99 (q, J=7.1 Hz, 2H), 3.74 (ddd, J=11.6, 5.8, 2.1 Hz, 1H), 3.63-3.56 (m, 1H), 3.45 (d, J=3.1 Hz, 1H), 3.36 (dt, J=7.1, 3.3 Hz, 4H), 3.24 (td, J=8.9, 4.7 Hz, 1H), 3.13 (d, J=4.8 Hz, 1H), 2.73 (t, J=6.2 Hz, 1H), 1.33 (t, J=7.0 Hz, 3H).

### Method 2:

Glycosyl donor 1 (37.5 mg, 0.15 mmol), 1-chloro-2-(4-ethoxybenzyl)-4-iodobenzene (0.1 mmol), Ru(bpy)₃Cl₂·6H₂O (0.8 mg, 1 mol %), NiBr₂·DME (3.1 mg, 10 mol %), diOMebpy (2.6 mg, 12 mol %), TMG (25 μL, 0.4 mmol), and DMSO (0.5 mL) were weighed, transferred into a vial with a spiral cap and a magnetic stirring bar, and then mixed. The vial was filled with N₂ and sealed with a Teflon cap. The mixture was stirred at room temperature under a 10 W, 455 nm LED light for 12 h to complete the reaction.

The reaction solution was diluted with 10 mL of ethyl acetate, and then washed with saturated NaCl solution. The organic phase was collected, and then the aqueous phase was extracted three times with 6 mL of ethyl acetate. The organic phases were combined and concentrated under reduced pressure. The residue was separated by silica gel chromatography (using a solution of petroleum ether/ethyl acetate=10:7 (v/v) as the eluent), to obtain the product dapagliflozin as pure (3-configuration, with a yield of 66% and a purity of >95%.

### Example 5: Preparation of Gliflozins Using Sodium Sulfinate Glycosyl Donors as Starting Materials: Empagliflozin

### Method 1:

By referring to the method described in Example 2, except that 4-methoxyiodobenzene was substituted with (3S)-3-[4-[(2-chloro-5-iodophenyl)methyl]phenoxy]tetrahydrofuran, the hypoglycemic drug empagliflozin was prepared as pure β-configuration, with a yield of 66% and a purity of >95%. The structural characterization was as follows:

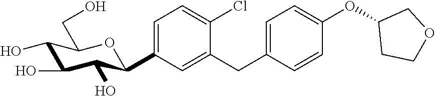

¹H NMR (400 MHz, CD₃OD) δ 7.37-7.31 (m, 2H), 7.27 (dd, J=8.1, 2.1 Hz, 1H), 7.13-7.06 (m, 2H), 6.81-6.74 (m, 2H), 4.94 (ddt, J=6.2, 4.1, 1.9 Hz, 1H), 4.08 (d, J=9.7 Hz, 1H), 4.05-3.95 (m, 2H), 3.94-3.80 (m, 5H), 3.71-3.64 (m, 1H), 3.48-3.34 (m, 3H), 3.30-3.24 (m, 1H), 2.19 (dtd, J=13.3, 8.4, 6.0 Hz, 1H), 2.10-2.02 (m, 1H).

### Method 2:

Glycosyl donor 1 (37.5 mg, 0.15 mmol), (3S)-3-[4-[(2-chloro-5-iodophenyl)methyl]phenoxy]tetrahydrofuran (41 mg, 0.1 mmol), Ru(bpy)₃Cl₂·6H₂O (0.8 mg, 1 mol %), NiBr₂·DME (3.1 mg, 10 mol %), diOMebpy (2.6 mg, 12 mol %), TMG (25 μL, 0.4 mmol), and DMSO (0.5 mL) were weighed, transferred into a vial with a spiral cap and a magnetic stirring bar, and then mixed. The vial was filled with N₂ and sealed with a Teflon cap. The mixture was stirred at room temperature under a 10 W, 455 nm LED light for 12 h to complete the reaction.

The reaction solution was diluted with 10 mL of ethyl acetate, and then washed with saturated NaCl solution. The organic phase was collected, and then the aqueous phase was extracted three times with 6 mL of ethyl acetate. The organic phases were combined and concentrated under reduced pressure. The residue was separated by silica gel chromatography (using a solution of petroleum ether/ethyl acetate=10:7 (v/v) as the eluent), to obtain the product empagliflozin as pure β-configuration, with a yield of 54% and a purity of >95%.

### Example 6: Preparation of Gliflozins Using Sodium Sulfinate Glycosyl Donors as Starting Materials: Ipragliflozin

### Method 1:

By referring to the method described in Example 2, except that 4-methoxyiodobenzene was substituted with 2-[(5-bromo-2-fluorophenyl)methylbenzothiophene, the hypoglycemic drug ipragliflozin was prepared as pure β-configuration, with a yield of 54% and a purity of >95%. The structural characterization was as follows:

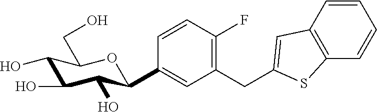

¹H NMR (400 MHz, CD₃OD) δ 7.72 (dd, J=8.1, 1.2 Hz, 1H), 7.69-7.60 (m, 1H), 7.43 (dd, J=7.4, 2.2 Hz, 1H), 7.35 (ddd, J=8.5, 5.0, 2.3 Hz, 1H), 7.32-7.19 (m, 2H), 7.12-7.02 (m, 2H), 4.34-4.18 (m, 2H), 4.11 (d, J=9.4 Hz, 1H), 3.91-3.82 (m, 1H), 3.73-3.63 (m, 1H), 3.48-3.35 (m, 3H), 3.33-3.31 (m, 1H).

### Method 2:

Glycosyl donor 1 (37.5 mg, 0.15 mmol), 2-[(5-bromo-2-fluorophenyl) methylbenzothiophene (0.1 mmol), Ru(bpy)₃Cl₂·6H₂O (0.8 mg, 1 mol %), NiBr₂·DME (3.1 mg, 10 mol %), diOMebpy (2.6 mg, 12 mol %), TMG (25 μL, 0.4 mmol), and DMSO (0.5 mL) were weighed, transferred into a vial with a spiral cap and a magnetic stirring bar, and then mixed. The vial was filled with N₂ and sealed with a Teflon cap. The mixture was stirred at room temperature under a 10 W, 455 nm LED light for 12 h to complete the reaction.

The reaction solution was diluted with 10 mL of ethyl acetate, and then washed with saturated NaCl solution. The organic phase was collected, and then the aqueous phase was extracted three times with 6 mL of ethyl acetate. The organic phases were combined and concentrated under reduced pressure. The residue was separated by silica gel chromatography (using a solution of petroleum ether/ethyl acetate=10:7 (v/v) as the eluent), to obtain the product ipragliflozin as pure (3-configuration, with a yield of 57% and a purity of >95%.

The following examples 7-10 are specific examples for preparation of gliflozins using a flow chemistry device.

### Example 7: Preparation of Gliflozins Using a Flow Chemistry Device

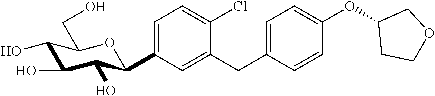

Glycosyl donor 1 (375 mg, 1.5 mmol), (3s)-3-[4-[(2-chloro-5-iodophenyl)methyl]phenoxy]tetrahydrofuran (410 mg, 1.0 mmol), Ru(bpy)₃Cl₂·6H₂O (8 mg), NiBr₂·DME (31 mg), diOMebpy (26 mg), TMG (250 μL) and DMSO (10 mL) were weighed and added into a vial with a spiral cap and a magnetic stirring bar, which was also pre-filled with N₂, and then mixed. The reaction solution was stirred to dissolve, so as to obtain a clear solution, which was transferred to a 10 mL injector. The injector was placed in a syringe pump, and the parameters were set up (flow rate 3 mL/h, and 0 means 0.3 mmol/h), while the other end of the injector was connected to a polytetrafluoroethylene (PTFE) tube, with a total volume of 3 mL and a inner diameter of 8 mm. The other end of the tube was linked to a collection bottle. The tube was illuminated under a 10 W, 455 nm LED lamp at room temperature, and then the syringe pump was turned on, so that the reaction solution was allowed to flow from the injector into the tube for irradiation. After 1 h of irradiation, the reaction solution per unit volume flew out into the collection bottle. After 4.3 h, all the reaction solution passed through the tube and into the collection bottle.

The reaction solution in the collection bottle was diluted with 30 mL of ethyl acetate, and then washed with saturated saline. The organic phase was collected, and then the aqueous phase was extracted three times with 30 mL of ethyl acetate. The organic phases were combined and concentrated under reduced pressure. The residue was separated by silica gel chromatography (using a solution of petroleum ether/ethyl acetate=10:7 (v/v) as the eluent) to obtain the glycoside product empagliflozin as pure β-configuration, with a yield of 54% and a purity of >95%.

¹H NMR (400 MHz, CD₃OD) δ 7.37-7.31 (m, 2H), 7.27 (dd, J=8.1, 2.1 Hz, 1H), 7.13-7.06 (m, 2H), 6.81-6.74 (m, 2H), 4.94 (ddt, J=6.2, 4.1, 1.9 Hz, 1H), 4.08 (d, J=9.7 Hz, 1H), 4.05-3.95 (m, 2H), 3.94-3.80 (m, 5H), 3.71-3.64 (m, 1H), 3.48-3.34 (m, 3H), 3.30-3.24 (m, 1H), 2.19 (dtd, J=13.3, 8.4, 6.0 Hz, 1H), 2.10-2.02 (m, 1H).

### Example 8: Preparation of Canagliflozin Using a Flow Chemistry Device

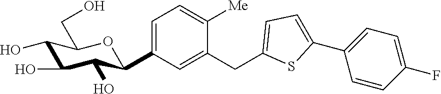

By referring to the method described in Example 7, the differences only laid in substituting (3s)-3-[4-[(2-chloro-5-iodophenyl)methyl]phenoxy]tetrahydrofuran with 2-(4-fluorophenyl)-5-[(5-iodo-2-methylphenyl)methyl]thiophene, as well as modifying the volume ratio of petroleum ether/ethyl acetate to 10:5 for the eluent of silica gel chromatography separation, and thereby the product canagliflozin was obtained as pure j-configuration, with a yield of 54% and a purity of >95%.

¹H NMR (400 MHz, CD₃CN) δ 7.37 (d, J=8.2 Hz, 1H), 7.30 (d, J=2.1 Hz, 1H), 7.24 (dd, J=8.2, 2.2 Hz, 1H), 7.12 (d, J=8.6 Hz, 2H), 6.86-6.80 (m, 2H), 4.08 (d, J=9.4 Hz, 1H), 4.05-4.02 (m, 2H), 3.99 (q, J=7.1 Hz, 2H), 3.74 (ddd, J=11.6, 5.8, 2.1 Hz, 1H), 3.63-3.56 (m, 1H), 3.45 (d, J=3.1 Hz, 1H), 3.36 (dt, J=7.1, 3.3 Hz, 4H), 3.24 (td, J=8.9, 4.7 Hz, 1H), 3.13 (d, J=4.8 Hz, 1H), 2.73 (t, J=6.2 Hz, 1H), 1.33 (t, J=7.0 Hz, 3H).

### Example 9: Preparation of Dapagliflozin Using a Flow Chemistry Device

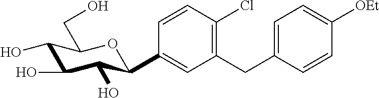

By referring to the method described in Example 7, except for substituting (3s)-3-[4-[(2-chloro-5-iodophenyl)methyl]phenoxy]tetrahydrofuran with 1-chloro-2-(4-ethoxybenzyl)-4-iodobenzene, the product dapagliflozin was obtained as pure 3-configuration, with a yield of 66% and a purity of >95%.

¹H NMR (400 MHz, CD₃CN) δ 7.37 (d, J=8.2 Hz, 1H), 7.30 (d, J=2.1 Hz, 1H), 7.24 (dd, J=8.2, 2.2 Hz, 1H), 7.12 (d, J=8.6 Hz, 2H), 6.86-6.80 (m, 2H), 4.08 (d, J=9.4 Hz, 1H), 4.05-4.02 (m, 2H), 3.99 (q, J=7.1 Hz, 2H), 3.74 (ddd, J=11.6, 5.8, 2.1 Hz, 1H), 3.63-3.56 (m, 1H), 3.45 (d, J=3.1 Hz, 1H), 3.36 (dt, J=7.1, 3.3 Hz, 4H), 3.24 (td, J=8.9, 4.7 Hz, 1H), 3.13 (d, J=4.8 Hz, 1H), 2.73 (t, J=6.2 Hz, 1H), 1.33 (t, J=7.0 Hz, 3H).

### Example 10: Preparation of Ipragliflozin Using a Flow Chemistry Device

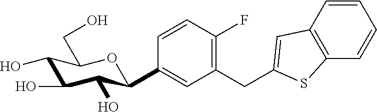

By referring to the method described in Example 7, except for substituting (3s)-3-[4-[(2-chloro-5-iodophenyl)methyl]phenoxy]tetrahydrofuran with 2-[(5-bromo-2-fluorophenyl) methylbenzothiophene, the product ipragliflozin was obtained as pure j-configuration, with a yield of 56% and a purity of >95%.

¹H NMR (400 MHz, CD₃OD) δ 7.72 (dd, J=8.1, 1.2 Hz, 1H), 7.69-7.60 (m, 1H), 7.43 (dd, J=7.4, 2.2 Hz, 1H), 7.35 (ddd, J=8.5, 5.0, 2.3 Hz, 1H), 7.32-7.19 (m, 2H), 7.12-7.02 (m, 2H), 4.34-4.18 (m, 2H), 4.11 (d, J=9.4 Hz, 1H), 3.91-3.82 (m, 1H), 3.73-3.63 (m, 1H), 3.48-3.35 (m, 3H), 3.33-3.31 (m, 1H).

The following Example 11 was a screening experiment for preparing gliflozins using a flow chemistry device.

### Example 11: Screening Experiment for Preparing Gliflozins Using a Flow Chemistry Device

Taking the preparation of dapagliflozin as an example, in the present invention, screening experiments on the types and amounts of raw materials and reaction solvents were carried out, using a flow chemistry apparatus. The specific experimental procedures were as follows:

### 1. Selection of Photosensitizer Types

By referring to the method of Example 9, the only difference was that the photosensitizer Ru(bpy)₃Cl₂·6H₂O was substituted with 14 other photosensitizers shown in Table 3, and then the yield of the product dapagliflozin obtained under the action of 14 other photosensitizers was calculated. The results are shown in Table 3.

TABLE 3
The yield of the product dapagliflozin obtained
under the action of 14 other photosensitizers.

| Entry | PC | Yield (%) |
|---|---|---|
| 1 | PC-1: Eosin Y/Na⁺ | 66 |
| 2 | PC-2: Mes-Acr⁺ CIO₄⁻ | 42 |
| 3 | PC-3: Eosin Y | 50 |
| 4 | PC-4: Rose bengal | 59 |
| 5 | PC-5: DPZ | 56 |
| 6 | PC-6: VB₂ | 20 |
| 7 | PC-7: 4CzIPN | 55 |
| 8 | PC-8: Rhodamine B | 45 |
| 9 | PC-9: Rhodamine 6G | 33 |
| 10 | PC-10: LuMiflavin | 30 |
| 11 | PC-11: fac-Ir(ppy)₃ | 40 |
| 12 | PC-12: Ru(phen)₃Cl₂•xH₂O | 46 |
| 13 | PC-13: Ru(phen)₃(PF₆)₂ | 54 |
| 14 | PC-14: Ru(Bphen)₃Cl₂ | 13 |

As shown, dapagliflozin could also be successfully prepared using 14 other photosensitizers listed in Table 3. Further combining with Example 9, it was found that when the photosensitizer was Ru(bpy)₃Cl₂·6H₂O or Eosin Y/Na⁺, the yield of the obtained product was the highest, and up to 6600.

### 2. Selection of the Amounts of Ni(II) Catalysts and Ligands

By referring to the method of Example 9, the only difference was to change the amounts of Ni(I) catalyst NiBr₂·DME and ligand diOMebpy, while the equivalent ratio of NiBr₂DME to diOMebpy was maintained to be 1:1.2 and unchanged. After the change, the amount of NiBr₂DME is shown in Table 4, the yield of the product dapagliflozin obtained was calculated in the presence of different amounts of Ni(II) catalysts and ligands. The results are shown in Table 4

TABLE 4
The yield of dapagliflozin obtained with different
amounts of Ni(II) catalysts and ligands.

| | Calculated based on the amount | |
|---|---|---|
| | of NiBr₂•DME (Maintaining | |
| | the equivalent ratio of NiBr₂•DME | |
| Entry | to diOMebpy to be 1:1.2) | Yield(%) |
| 1 | 10% | 66% |
| (Example 9) | | |
| 2 | 5% | 64% |
| 3 | 4% | 59% |
| 4 | 3% | 39% |
| 5 | 2% | 28% |
| 6 | 1% | 43% |

As shown, when the ligand amount was 10%, the yield of the product obtained was the highest, and up to 66%.

### 3. Selection of Ni(II) Catalyst Types

Referring to the method of Example 9, the only difference was from replacing the Ni(II) catalyst NiBr₂·DME with nickel bromide, nickel chloride, nickel acetate, and nickel acetylacetonate shown in Table 5, respectively, and then the yield of dapagliflozin obtained in the presence of other Ni(II) catalysts was calculated. The results are shown in Table 5.

TABLE 5
Yield of dapagliflozin obtained in the
presence of other Ni(II) catalysts.

| Entry | Ni(II) catalysts | Yield |
|---|---|---|
| 1 | NiBr₂•DME | 66% |
| (Example 9) | | |
| 2 | NiBr₂ | 64% |
| 3 | NiCl₂ | 50% |
| 4 | Ni(OAc)₂ | 46% |
| 5 | Ni(acac)₂ | 33% |

As shown, when NiBr₂·DME was used as the catalyst, the highest yield of the product was obtained, and up to 66%.

### 4. Selection of Solvent Types

By referring to the method of Example 9, the only difference was that the solvent was changed from DMSO to DMF and DMA, as shown in Table 6, and the yield of the product dapagliflozin obtained by the reaction in the solvent was calculated. The results are shown in Table 6.

TABLE 6
Yield of the product dapagliflozin obtained in different solvents.

| Entry | Solvent | Yield (%) |
|---|---|---|
| 1 | DMSO | 66% |
| (Example 9) | | |
| 2 | DMF | 28% |
| 3 | DMA | 30% |

As shown, when the solvent was DMSO, the yield of the obtained product was the highest, and up to 66%.

In summary, aryl glycosides including gliflozins were prepared for the first time using the sulfinate glycosyl donor of the present invention as the starting materials. This method did not require additional initiators; featured low production costs, a simple reaction route, mild reaction conditions, high product yield and purity; and showed broad application prospects.

1. A method for preparing aryl C-glycosides, characterized in that the method comprises the following steps: a glycosyl donor represented by formula W, a glycosyl acceptor represented by formula B-1, Ni(II) catalyst, a photosensitizer, a ligand, and a base are added to a solvent, and then allowed to react under light, to obtain an aryl C-glycoside represented by formula B-2; 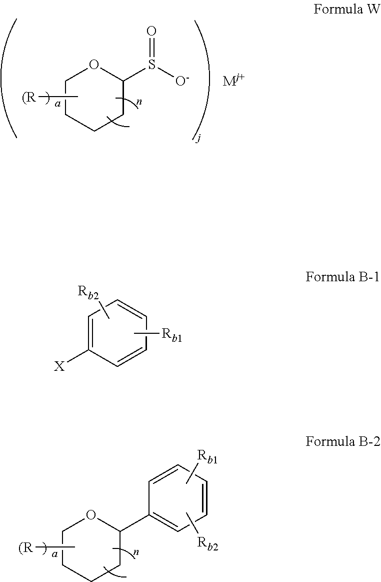
wherein, n is selected from 0 or 1;
a is selected from 3 or 4;
R is each independently selected from L₁Rₓ; alternatively, two adjacent R are linked to form a ring, and additional R is independently selected from L₁Rₓ, wherein the ring is unsubstituted or substituted with one or more L₁Rₓ;
L₁ is selected from absence or C₁₋₂ alkylene; Rx is selected from the group consisting of H, hydroxyl, C₁₋₆ alkyl, OAc, OBn, OR₈, NR₉R₁₀, 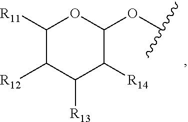
Ph, amino, 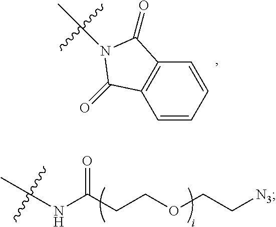
i is an integer selected from 0 to 6;
R₁₁, R₁₂, R₁₃, and R₁₄ are each independently selected from L₂R_{y}; L₂ is selected from absence or C₁₋₂ alkylene, and R_{y} is selected from the group consisting of H, hydroxyl, C₁₋₆ alkyl, OAc, OBn, OR₈, and NR₉R₁₀;
R₈ is selected from C₁₋₆ alkyl;
R₉ is selected from the group consisting of H, C₁₋₆ alkyl, Ac, and Bn;
R₁₀ is selected from the group consisting of H, C₁₋₆ alkyl, Ac, and Bn;
j is 1, 2, or 3;
M^{j+} is a j-valent cation;
X is halogen;
R_{b1} is selected from the group consisting of H, hydroxyl, C₁₋₆ alkyl, C₁₋₆ alkoxy, and halogen;
R_{b2} is selected from the group consisting of H and 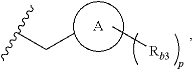
wherein ring A is selected from the group consisting of 5-6-membered aryl, 5-6 membered heteroaryl, fused cycloalkyl, or fused heterocyclic group; p is an integer from 0 to 2; R_{b3} is each independently selected from the group consisting of C₁₋₄ alkyl, C₁₋₄ alkoxy, halogenated or unhalogenated phenyl, and OR_{b4}, and R_{b4} is selected from 5-6-membered saturated heterocyclic group or 5-6 membered saturated cycloalkyl.

2. The method according to claim 1, characterized in that the structure of the glycosyl donor is as represented by formula I: 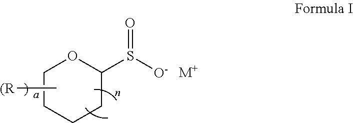
wherein, n is selected from 0 or 1;
a is selected from 3 or 4;
R is each independently selected from L₁Rₓ; alternatively, two adjacent R are linked to form a ring, and the remaining R is independently selected from L₁Rₓ, wherein the ring is unsubstituted or substituted with one or more L₁Rₓ;
L₁ is selected from absence or C₁₋₂ alkylene; Rx is selected from the group consisting of H, hydroxyl, C₁₋₆ alkyl, OAc, OBn, OR₈, NR₉R₁₀, 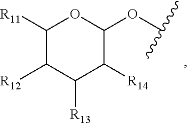
Ph, amino, 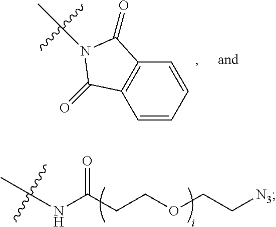
i is an integer selected from 0 to 6;
R₁₁, R₁₂, R₁₃, and R₁₄ are each independently selected from L₂R_{y}; L₂ is selected from absence or C₁₋₂ alkylene, and R_{y} is selected from the group consisting of H, hydroxyl, C₁₋₆ alkyl, OAc, OBn, OR₈, and NR₉R₁₀;
R₈ is selected from C₁₋₆ alkyl;
R₉ is selected from the group consisting of H, C₁₋₆ alkyl, Ac, and Bn;
R₁₀ is selected from the group consisting of H, C₁₋₆ alkyl, Ac, and Bn;
M^{j+} is a monovalent cation.

3. The method according to claim 2, characterized in that the structure of the glycosyl donor is as represented by formula II: 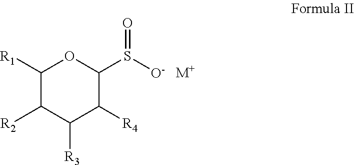
wherein, R₁, R₂, R₃, and R₄ are each independently selected from L₁Rₓ; alternatively, for R₁, R₂, R₃, and R₄, two adjacent groups are linked to form a ring, and two remaining groups are each independently selected from L₁Rₓ. The ring is a 5-6-membered ring which is unsubstituted or substituted with one or more L₁Rₓ;
L₁ is selected from absence or methylene, and Rₓ is selected from the group consisting of H, OH, C₁₋₅ alkyl, OAc, OBn, OR₈, NR₉R₁₀, 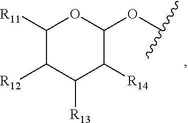
Ph, amino, 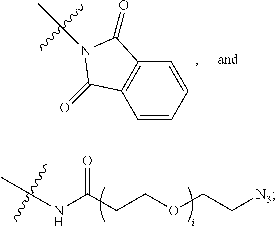
i is an integer selected from 0 to 4;
R₁₁, R₁₂, R₁₃, and R₁₄ are each independently selected from L₂R_{y}; L₂ is selected from absence or methylene, and R_{y} is selected from the group consisting of H, hydroxyl, C₁₋₅ alkyl, OAc, OBn, OR₈, and NR₉R₁₀;
R₈ is selected from C₁₋₅ alkyl;
R₉ is selected from the group consisting of H, C₁₋₅ alkyl, Ac, and Bn;
R₁₀ is selected from the group consisting of H, C₁₋₅ alkyl, Ac, and Bn;
M^{j+} is a monovalent cation.

4. The method according to claim 2, characterized in that the structure of the glycosyl donor is as represented by formula III: 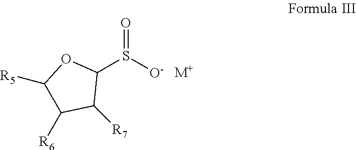
wherein, R₅, R₆, and R₇ are each independently selected from L₁Rₓ; alternatively, for R₅, R₆, and R₇, two adjacent groups are linked to form a ring, and the remaining group is L₁Rₓ. The ring is a 5-6-membered ring that is unsubstituted or substituted with one or more L₁Rₓ;
L₁ is selected from absence or methylene, and Rₓ is selected from the group consisting of H, OH, C₁₋₅ alkyl, OAc, OBn, OR₈, NR₉R₁₀, 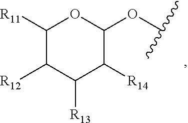
Ph, amino, 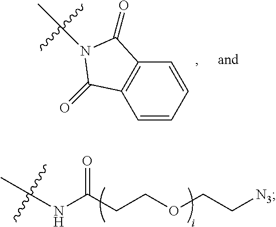
i is an integer selected from 0 to 4;
R₁₁, R₁₂, R₁₃, and R₁₄ are each independently selected from L₂R_{y}; L₂ is selected from absence or methylene, and R_{y} is selected from the group consisting of H, hydroxyl, C₁₋₅ alkyl, OAc, OBn, OR₈, and NR₉R₁₀;
R₈ is selected from C₁₋₅ alkyl;
R₉ is selected from the group consisting of H, C₁₋₅ alkyl, Ac, and Bn;
R₁₀ is selected from the group consisting of H, C₁₋₅ alkyl, Ac, and Bn;
M^{j+} is a monovalent cation.

5. The method according to claim 3, characterized in that the 5-6-membered ring is a 5-6-membered saturated oxygen-containing heterocycle;
R₈ is selected from C₁₋₃ alkyl;
R₉ is selected from the group consisting of H, C₁₋₃ alkyl, Ac, and Bn;
R₁₀ is selected from the group consisting of H, C₁₋₃ alkyl, Ac, and Bn;
M⁺ is selected from the group consisting of Na⁺, K⁺, and Li⁺.

6. The method according to claim 1, characterized in that the structure of the glycosyl donor is selected from the group consisting of:

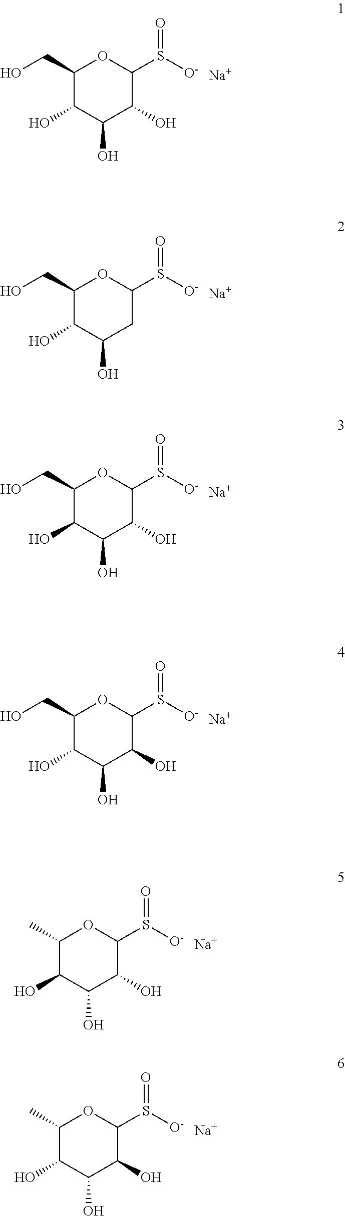

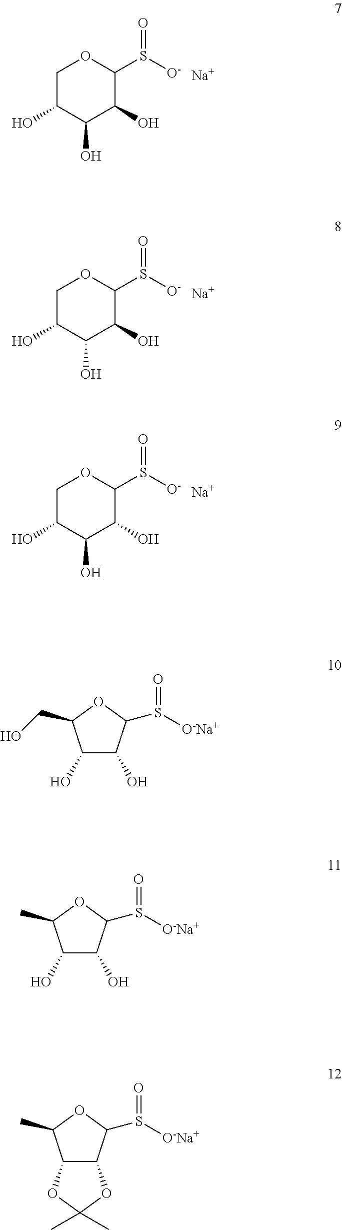

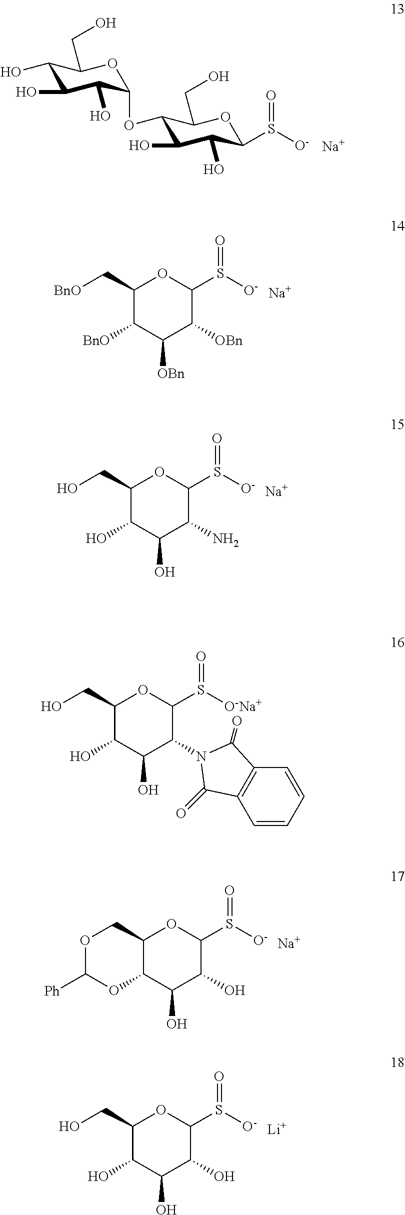

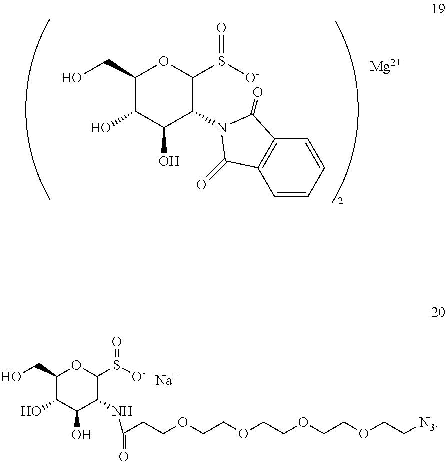

7. The method according to claim 1, characterized in that the molar ratio of the glycosyl donor, glycosyl acceptor represented by formula B-1, photosensitizer, Ni (II) catalyst, ligand, and base is (0.07-0.3):(0.05-0.2):(5-20):(50-200):(10-240):(0.1-0.4); preferably 0.15:0.1:10:100:(50-120):0.2; and more preferably 0.15:0.1:10:100:120:0.2;
and/or, said photosensitizer is selected from Ru(II) photosensitizers, eosin Y or salts thereof, wherein the Ru (II) photosensitizer is preferably Ru(bpy)₃Cl₂·6H₂O, and the salt of eosin Y is preferably Eosin Y/Na⁺;
and/or, said Ni (II) catalyst is NiBr₂·DME, NiBr₂, NiCl₂, Ni(OAc)₂ or Ni(acac)₂, and preferably NiBr₂·DME;
and/or, said ligand is N,N ligand, and preferably diOMebpy;
and/or, said base is an organic or inorganic base, and preferably tetramethylguanidine;
and/or, said solvent is an organic solvent; preferably DMSO, DMF, and DMA; and more preferably DMSO;
and/or, the reaction is carried out under an inert gas atmosphere; the reaction temperature is room temperature, and the reaction time is 4-14 h;
and/or, said X is iodine or bromine.

8. The method according to claim 1, characterized in that the aryl C-glycosides represented by formula B-2 are 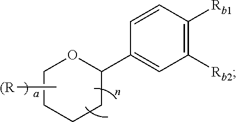
R_{b1} is selected from the group consisting of H, C₁₋₃ alkyl, C₁₋₃ alkoxy, and halogen; R_{b2} is selected from the group consisting of H,

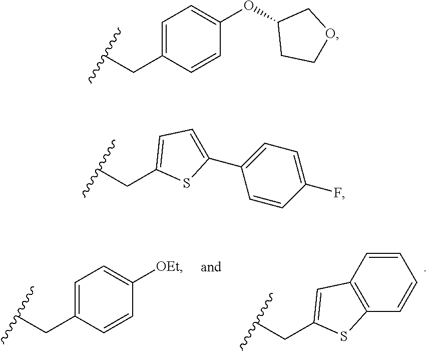 [SMILES: *Cc1ccc(O[C@H]2CCOC2)cc1 ; *Cc1ccc(-c2ccc(F)cc2)s1 ; *Cc1ccc(OCC)cc1 ; *Cc1cc2ccccc2s1]

9. The method according to claim 8, characterized in that the aryl C-glycosides represented by formula B-2 are selected from the group consisting of:

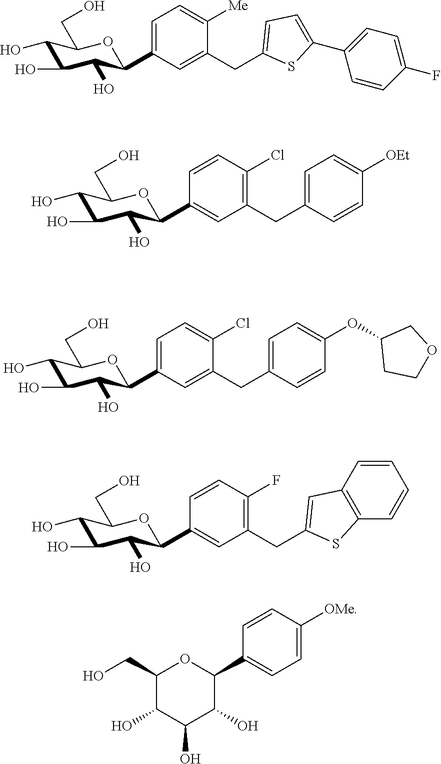

10. The method according to claim 1, characterized in that the reaction device is that for a stirring reaction or flow chemistry.
